(19) United States
(12) Patent Application Publication
(10) Pub. No.: US 2007/0213731 A1
(43) Pub. Date: Sep. 13, 2007

(54) Pedicle Screws for Osteoporosis

(76) Inventor: Chad J. Prusmack, Denver, CO (US)

Correspondence Address: GLOBUS MEDICAL, INC.;ATTN: BRIAN MALM, VALLEY FORGE BUSINESS CENTER, 2560 GENERAL ARMISTEAD AVENUE, AUDUBON, PA, 19403, US

(21) Appl. No.: 11/683,389

(22) Filed: Mar. 7, 2007

Related U.S. Application Data
(60) Provisional application No. 60/779,794, filed on Mar. 7, 2006.

(51) Int. Cl. A61B 17/58

(52) U.S. Cl. 606/73

(57) ABSTRACT

A bone screw for osteoporosis is disclosed.

### CROSS-REFERENCE TO RELATED APPLICATIONS

The present application claims priority to U.S. Provisional Application No. 60/779,794, filed Mar. 7, 2006, the entire contents of which are incorporated herein by reference.

### BACKGROUND OF THE INVENTION

In spine fusion surgery, commonly, instrumentation is used to:

1. Stabilize the spine.

2. Increase fusion rates for better patient outcome.

Instrumentation commonly used for spinal fusion procedures are pedicle screws and rods. Typically, pedicle screws are placed in through the pedicle into the body of adjacent vertebrae and then connected with rods to secure the segment between two adjacent vertebrae to eventually fuse them. However, important principles for successful fusion involve the screw's material strength, screw pull-out strength and the bone/screw interface strength. These principles must provide the appropriate mechanical support between the bone, screw, rod, and adjacent segment for the fusion not to ‘fail’. Osteoporosis is a common reason for failure with instrumentation because the bone/screw interface is poor. A poor interface allows for screws to pull out, cause fractures, and/or become loose and eventually fail.

### BRIEF DESCRIPTION OF THE DRAWINGS

The invention will be more readily understood with reference to the embodiments thereof illustrated in the attached figures, in which:

FIGS. 1-6 are views of one embodiment of a method of inserting a bone screw according to the invention;

FIGS. 7, 7A and 7B are views of one embodiment of a bone screw according to the invention;

FIGS. 8 and 8A are views of another embodiment of a bone screw according to the invention; and

FIGS. 9, 9A and 9B are views of another embodiment of a bone screw according to the invention.

### DETAILED DESCRIPTION OF THE INVENTION

Certain embodiments of the present invention involve a novel technique which may utilize one of three uniquely designed types of minimally invasive pedicle screws which could be meant for patients with osteoporosis utilizing an augmentation technique with methyl methacrylate.

One embodiment of a technique according to the invention begins similar to a vertebroplasty in which cement maybe injected into the vertebral body and pedicle first, before the screws are placed. Then, utilizing the present technique, the screws, rather than being placed in the soft bone, are placed in a diffuse morass of methyl methacrylate, which will cover a larger volume of the vertebra and fill the screws thereby improving the mechanical properties of the bone screw interface. Because of the particular technical modification of these unique screws, they may also be able to accept the methyl methacrylate better and therefore provide a novel technique for fusions in patients who are osteoporotic.

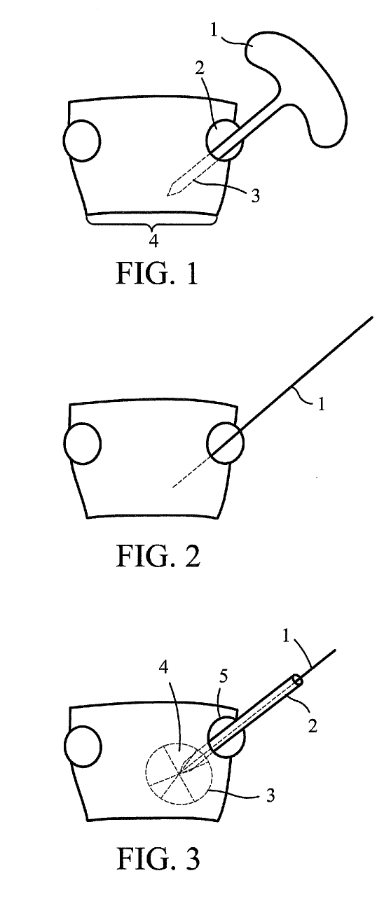

One type of instrumented fusion can be done with cannulated percutaneous pedicle screws. Percutaneous pedicle screws are placed using an already described technique first using a cannulated bone biopsy needle. FIGS. 1-6 show one embodiment of a method according to the invention and FIGS. 1-3 show a vertebra in an anterior-posterior radiographic view. FIG. 1 shows a cannulated introducer going into the pedicle and the body of the vertebra 3. The inner cannula is removed, and a guidewire is placed through the cannulated needle, and the needle is removed. In FIG. 2, a guidewire remains in the pedicle and the body. At this point, the surgeon could gauge whether or not the bone was “too soft” for a regular pedicle screw system and then would have the ‘option’ at that moment in surgery to utilize the present technique.

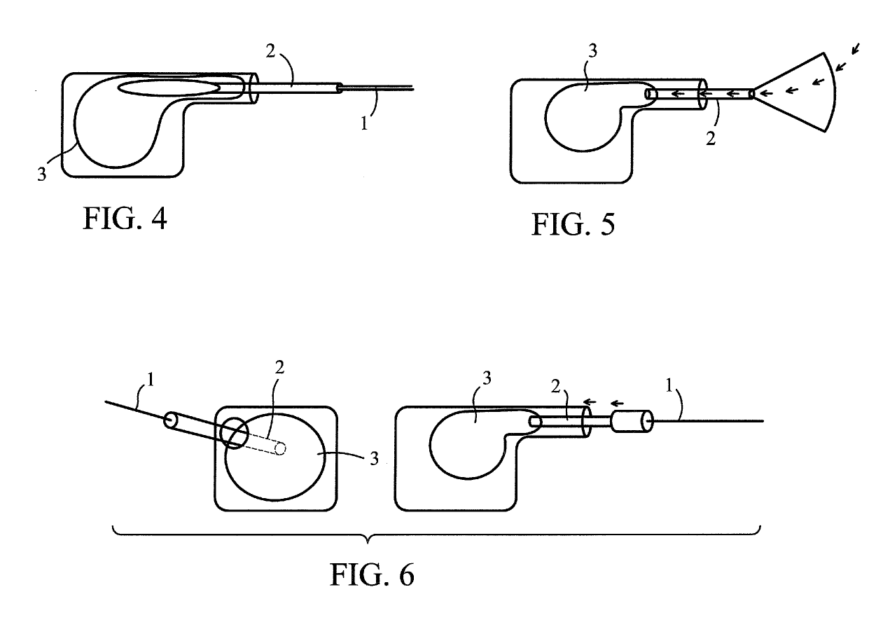

With reference to FIG. 3, one embodiment of the technique may include placing a gouge or balloon as an instrument 2, over the guidewire 1. This gouge or balloon would create more space 4, in the soft bone and open up space 3 and 5, both in the body and the pedicle (FIG. 3). FIG. 4 shows a lateral view of the gouge which may be expandable and in a brush-like configuration 2. It could be moved in an in-and-out fashion through the pedicle 1. This would then create an empty space both in the pedicle and the body 3 in FIG. 4. Referring to FIG. 5, the introduction of methyl methacrylate through an introducer 2 into this evacuated space 3 is shown. Therefore, the methyl methacrylate may fully fill the open area 1 both in the pedicle and in the body.

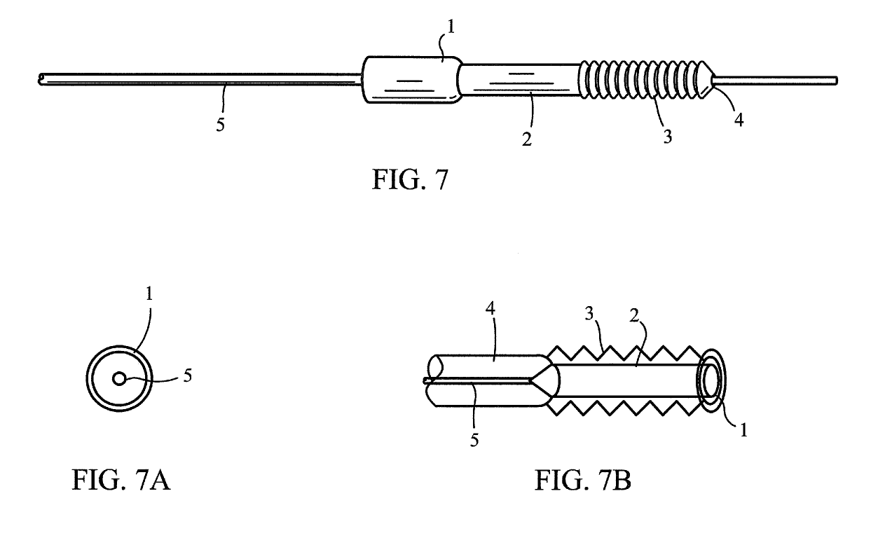

At this point, with a large volume of the pedicle and body of the osteoporotic vertebra filled with methyl methacrylate, one would then introduce a screw into the methyl methacrylate as shown in FIG. 6. One embodiment of a screw could be cannulated to go over the guidewire 1 and then submerged into the methyl methacrylate 2 in both the anterior-posterior and lateral versions. The screw may be angled medially into the vertebral body and/or methyl methacrylate 3 in both figures. Types of screws believed to be particularly accepting and mechanically strong when the methyl methacrylate dried are described in FIGS. 7, 8, and 9.

Referring to FIG. 7, one embodiment of a screw according to the invention is shown as being generally hollow tipped. This screw is generally able to fit over a guidewire 5, has a polyaxial screw head for the titanium rod fixation 1 and has a solid mid shaft 2 to provide a strong interface between the hollow end 3 and the polyaxial screw 1. The area 3 preferably has regular screw etches or threads, but it is hollow. Therefore hollow tip 4 provides a reservoir for the methyl methacrylate to fill when the guidewire 5 is removed. This provides a strengthened cement screw interface. Referring to FIG. 7B, a cross-sectional view is shown, with the hollow tip 1 configured to accept the methyl methacrylate, which fills the areas 2 and 5. The strength of the screw is maintained by the solid features 3 and 4. FIG. 7A depicts the front view of the hollow tip with the sharp rim.

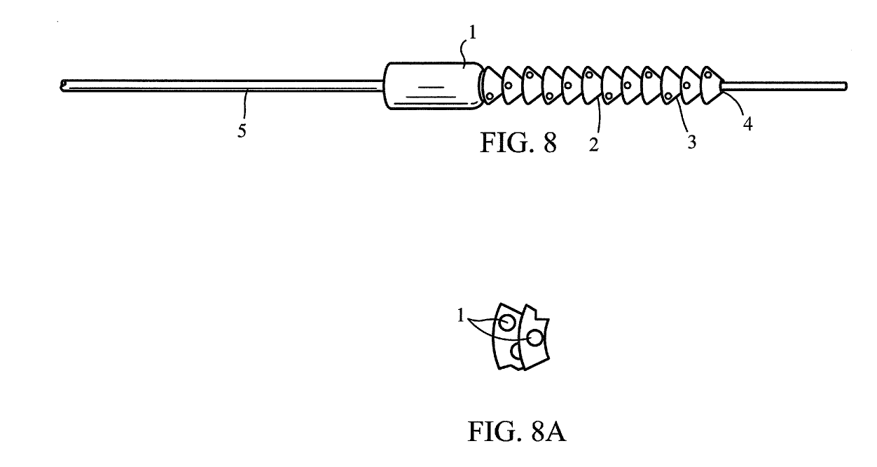

Referring to FIG. 8, another embodiment of a dimpled screw is shown. The polyaxial screw head 1, a longer dimpled screw system 2 has indentations 3 that make the interface between the methyl methacrylate and the screw stronger by increasing the coefficient of friction or defining interference surface features such that it possesses a stronger pull out strength. More closely looking at the dimples of FIG. 8A, one can see that they may be indented, but in one embodiment they do not go through the full thickness of the titanium screws into the area which is cannulated for the guidewire labeled 4. This provides extra strength to the screw because of more metal being available to endure the stresses.

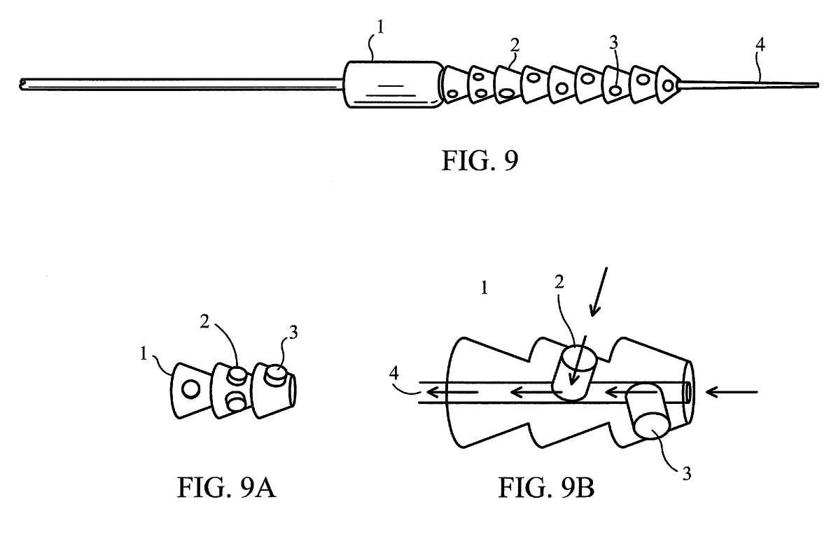

As illustrated in FIG. 9, another embodiment of a porous-type screw is shown. In this embodiment, the screw has a polyaxial screw head 1, and a similar type of screw end 2. However, preferably the screw has full cannulated pores 3 instead of dimples. As seen in FIG. 9A, the pores 2 are in communication with the area utilized for the guidewire 3. Therefore, after inserting it into the methyl methacrylate, the methyl methacrylate could then enter the pores and form a continuum, as seen in FIG. 9B. In use, methyl methacrylate may enter areas 5, 1, 3, 1, and 2 and meet in continuity in 4 and therefore result in a solid interface between the screw and methyl methacrylate and osteoporotic bone. A suction may be applied to area 4 on the screw head 1 to facilitate absorption of methacrylate.

In summary, the foregoing is a novel technique which can be employed in the middle of the fusion procedure as a result of the surgeon's discretion thinking that his bone he was working with was too osteoporotic. The surgeon could then utilize a more diffuse methyl methacrylate interface between the bone and instrumentation and preferably a second more advantageous interface between the methyl methacrylate and the unique type of screws designated hollow tipped, dimpled, and porous. This would then allow applicability to osteoporotic patients so they could have fusion safer and more frequently for their poor spine conditions.

What is claimed is:

1. A bone screw, comprising:
a cannulated body extending from a proximal end to a distal end and defining a central cannula;
a plurality of threads provided along at least a portion of the body; and
a plurality of pores extending through the body and into the central cannula.
